(19) United States
(12) Patent Application Publication
(10) Pub. No.: US 2026/0000456 A1
(43) Pub. Date: Jan. 1, 2026

(54) Systems and Methods for Endoluminal Tracking with Therapeutic Delivery Sensing

(71) Applicant: Frond Medical Inc., Fremont, CA (US)

(72) Inventors: Xin Lu, Palo Alto, CA (US); Rose Monaghan, Los Gatos, CA (US)

(21) Appl. No.: 18/869,143

(22) PCT Filed: Jun. 2, 2023
(86) PCT No.: PCT/US2023/067851

§ 371 Date: Nov. 25, 2024

Related U.S. Application Data
(60) Provisional application No. 63/365,770, filed on Jun. 2, 2022.

(51) Int. Cl. A61B 34/10; A61B 5/00; A61B 5/02; A61B 5/06; A61B 34/00; A61B 34/20; A61B 90/00

(52) U.S. Cl. CPC A61B 34/10; A61B 5/0036; A61B 5/02007; A61B 5/064; A61B 5/6853; A61B 5/7267; A61B 34/20; A61B 34/25; A61B 90/39; A61B 2034/104; A61B 2034/107; A61B 2034/2055; A61B 2090/3954; A61B 2090/3966; A61B 2562/0247

(57) ABSTRACT

Systems and methods are provided that can enable improved detection of a position within a body lumen at which a therapy is delivered and improved labelling of patient data sets. A system includes a first flexible elongate instrument comprising a plurality of imaging markers and a second flexible elongate instrument, comprising a therapeutic delivery device, configured for relative movement with respect to the first instrument. A treatment activation sensor is included to detect an activation energy associated with delivery of a therapy by the therapeutic delivery device. The system further includes a processor configured to establish a reference coordinate system based on the plurality of imaging markers, receive therapeutic delivery information at a plurality of locations in the body lumen from a location information sensor, and correlate the therapeutic delivery information with the detected activation energy to determine a position of the delivered therapy based on the reference coordinate system.

### RELATED APPLICATION

This application claims the benefit of U.S. Provisional Application No. 63/365,770, filed on Jun. 2, 2022. The entire teachings of the above application are incorporated herein by reference.

### BACKGROUND

Intracoronary imaging is often used to accurately measure vessel and stenosis dimensions, assess vessel integrity, characterize lesion morphology and aide in body lumen procedures, including percutaneous coronary intervention (PCI) procedures. The frequency of complex percutaneous coronary interventions has steadily increased in recent years due to clinical benefits provided by the interventions, which can increase the life expectancy and quality of life for patients suffering from endovascular neurosurgical, cardiovascular, and peripheral artery diseases. Various diagnostic and therapeutic medical devices (e.g., guidewires, balloons, atherectomy, lithotripsy, stents, imaging and physiology diagnostic modalities, X-ray angiography, and fluoroscopy) enable radiologists, cardiologists, and vascular specialists to visualize a patient's intra-vasculature to guide treatment decisions and to perform intervention procedures. Often, X-ray fluoroscopy with contrast injection is used to guide physicians to position devices (e.g., stents, guidewires, and balloons) toward targeted lesion locations along a guidewire within the endo-vasculature.

In a PCI procedure, vascular access is typically gained through an arterial entry point, such as the radial, brachial, or femoral artery, or through a venous puncture. From the entry point, a physician can access the vasculature of organs such as heart, lungs, kidneys, and brain by advancing a guidewire into the patient until a distal end of the guidewire crosses, for example, a lesion to be treated. After the guidewire position is finalized and situated such that it is viewable on an angiographic image, a desired therapeutic and/or diagnostic device is mounted on a proximal end of the guidewire. The therapeutic and/or diagnostic device is then advanced towards the distal end to the feature of interest.

Depending upon the clinical situation, imaging and/or physiological probes, such as Intravascular Ultrasound (IVUS), Optical coherence tomography (OCT) and Fractional Flow Reserve (FFR) devices, can be used for pre-intervention assessment, such as for determining lesion location, lesion dimension, plaque morphology, and coronary pressure at an area of interest. Endoluminal diagnostic modalities, such as IVUS, OCT, and FFR, which are able to generate more detailed vessel lumen information than that which can be obtained from X-ray imaging alone, are widely used for minimally invasive PCI procedures.

Endoluminal device guidance generally requires a live display of the device's movement inside of a body lumen. The methods currently available for guidance and positioning are based on real-time fluoroscopic imaging using X-ray, such that both a blood vessel's lumen path and the device inside of the lumen are continuously visible during the procedure. X-ray imaging for blood vessel diagnosis and device guidance emits X-rays at many frames per second and often requires contrast fluid injection, which allows for visualization of the vessel to help clinicians locate and position medical instruments. This practice results in high radiation exposure to both patients and clinicians, as well as the delivery of large volumes of contrast agents to patients, which are harmful to the kidneys.

Upon diagnostic assessment, interventional devices may then be guided to an area in need of treatment, which can also require guidance by fluoroscopic imaging. Positioning of an interventional device at a location identifiable on a diagnostic dataset can be difficult.

There exists a need for improved systems and methods for providing endoluminal device guidance and locating medical devices within a body lumen, particularly with respect to providing for accurate delivery of a treatment to a location within the lumen.

### SUMMARY

Systems and methods are provided that can enable improved detection of a position within a body lumen at which a therapy is delivered and improved labelling of patient data sets for treatment delivery locations with respect to anatomical data. With such improved position detection and labelling, systems and methods comprising machine learning can provide for informing improved treatment decisions and providing improved device guidance.

A system for locating a treatment provided in an endoluminal procedure includes a first flexible elongate instrument comprising a plurality of imaging markers and a second flexible elongate instrument configured for relative movement with respect to the first flexible elongate instrument. The second flexible elongate instrument comprises a therapeutic delivery device. The system further includes a location information sensor disposed at the first flexible elongate instrument or at the second flexible elongate instrument and a treatment activation sensor. The treatment activation sensor is configured to detect an activation energy associated with delivery of a therapy by the therapeutic delivery device. The system further includes a processor configured to establish a reference coordinate system based on the plurality of imaging markers, the plurality of imaging markers being visible in a medical image comprising the first flexible elongate instrument disposed in a body lumen. The processor is further configured to receive therapeutic delivery information at a plurality of locations in the body lumen from the location information sensor and correlate the therapeutic delivery information with the detected activation energy to determine a position of the delivered therapy within the body lumen based on the reference coordinate system.

The activation sensor can be pressure sensor, and the therapeutic delivery device can be an inflation device.

The processor can be further configured to receive diagnostic scan information at the plurality of locations in the body lumen from a diagnostic device and correlate the diagnostic scan information with the therapeutic delivery information and the detected activation energy to determine a position of the delivered therapy relative to the diagnostic scan information. Receipt of the diagnostic scan information can occur prior to delivery of the therapy. The first flexible elongate instrument can comprise the diagnostic device, or the diagnostic device can be configured for relative movement with respect to the first flexible elongate instrument.

The therapy can comprise an angioplasty, atherectomy, stent delivery, lithotripsy, or a combination thereof. The therapeutic delivery device can be a catheter or other device configured to deliver the provided therapy.

The system can further include a display configured to display a composite image comprising a representation of the position of the delivered therapy for use with subsequent procedures.

A method for locating a treatment provided in an endoluminal procedure includes establishing a reference coordinate system based on a plurality of imaging markers of a first flexible elongate instrument, the plurality of imaging markers being visible in a medical image comprising the first flexible elongate instrument disposed in a body lumen. The method further includes determining therapeutic delivery information at a plurality of locations in the body lumen from a location information sensor disposed at the first flexible elongate instrument or at a second flexible elongate instrument. The second flexible elongate instrument is configured for relative movement with respect to the first flexible elongate instrument and comprises a therapeutic delivery device. The method further includes detecting an activation energy associated with associated with delivery of a therapy by the therapeutic delivery device and correlating the therapeutic delivery information with the detected activation energy to determine a position of the delivered therapy within the body lumen based on the reference coordinate system.

Detecting the activation energy can comprise detecting a pressure, which can, for example, indicate inflation of the therapeutic delivery device.

The method can further include receiving diagnostic scan information at the plurality of locations in the body lumen from a diagnostic device and correlating the diagnostic scan information with the therapeutic delivery information and the detected activation energy to determine a position of the delivered therapy relative to the diagnostic scan information. Receiving the diagnostic scan information can occur prior to delivery of the therapy. The first flexible elongate instrument can comprise the diagnostic device, or the diagnostic device can be one that is configured for relative movement with respect to the first flexible elongate instrument.

The therapy can comprise an angioplasty, atherectomy, stent delivery, lithotripsy, or a combination thereof. The therapeutic delivery device can be a catheter or other device configured to deliver the provided therapy.

The method can further include displaying a composite image comprising a representation of the position of the delivered therapy for use with subsequent procedures.

A method for generating a treatment recommendation for an endoluminal procedure for a patient includes receiving diagnostic scan information at a plurality of locations in a body lumen of the patient, the diagnostic scan information correlated to a reference coordinate system established based on imaging markers of a flexible elongate instrument disposed in the body lumen and visible in a medical image of the body lumen. The method further includes applying a machine learning model to the diagnostic scan information to identify a prospective treatment location. The machine learning model is trained (and/or calibrated) using training data comprising diagnostic scan information and therapeutic delivery information, including detected activation information for the therapeutic delivery, for a set of patients. The method further includes outputting a treatment recommendation for the patient based on the applied machine learning model, the treatment recommendation including a location for treatment identified with respect to the reference coordinate system.

A method for generating a treatment recommendation for an endoluminal procedure for a patient includes receiving diagnostic scan information at a plurality of locations in a body lumen of the patient, the diagnostic scan information correlated to a reference coordinate system established based on imaging markers of a flexible elongate instrument disposed in the body lumen and visible in a medical image of the body lumen. The method further includes applying a machine learning model to the diagnostic scan information to obtain a predictive label associated with one or more treatment parameters. The machine learning model is trained (and/or calibrated) using training data comprising diagnostic scan information and therapeutic delivery information, including detected activation information for the therapeutic delivery, for a set of patients. The method further includes outputting a treatment recommendation for the patient based on the obtained predictive label.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of an example system for locating a medical device in a body lumen and providing for treatment delivery position detection.

FIG. 2 depicts simulated images generated from an example IVUS scan and example FFR scan with the benefit of an example co-location location system.

FIG. 3A is a schematic of an example device in which a flexible elongate instrument includes a sensor for detection of displacement encoding markers.

FIG. 3B is a schematic of an example encoding liner for use with the device of FIG. 3A.

FIG. 4 is a schematic of an example system in which displacement encoding markers of a flexible elongate instrument are detected for displacement measurement.

FIG. 5 is a schematic of an example system in which a flexible elongate instrument having an encoding sensor is used in conjunction with a therapeutic device delivering an angioplasty balloon.

FIG. 6 depicts simulated images generated from an example IVUS scan and example FFR scan with the benefit of an example co-location location system with treatment delivery sensing for delivery of stents.

FIG. 7 depicts simulated images generated from an example OCT scan with the benefit of an example co-location location system with treatment delivery sensing for intravascular lithotripsy (IVL).

FIG. 8 is a block diagram of an example co-location system with artificial intelligence for the training of a machine learning model and providing for predictive treatment recommendations.

FIG. 9 is a flow diagram of an example process for treatment labeling that can be used in a machine learning model.

### DETAILED DESCRIPTION

A description of example embodiments follows.

Examples of systems and methods providing for position detection of endoluminal instruments are described in International Pub. No. WO 2022/126101, titled “Methods and Systems for Body Lumen Medical Device Location,” the entire teachings of which are incorporated herein by reference.

Improvements to the systems and methods provided in Intl. Pub. No. WO 2022/126101 are provided. The systems and methods described herein can provide for improved detection of treatment location(s) involved in an endoluminal procedure with the inclusion of treatment delivery sensing, such as pressure sensing for the delivery of a balloon and/or lithotripsy. The inclusion of such sensing can also be employed in the labelling of patient data to better inform future treatment decisions (e.g., for a same patient, or for a new patient) by way of a machine learning model trained on prior labeled datasets and providing for predictive treatment locations and treatment types.

The example devices, systems and methods described herein are generally described within the context of percutaneous coronary intervention (PCI) procedures; however, the provided devices and systems can be applied to or used within the context of other types of endoluminal procedures, such as gastrointestinal procedures.

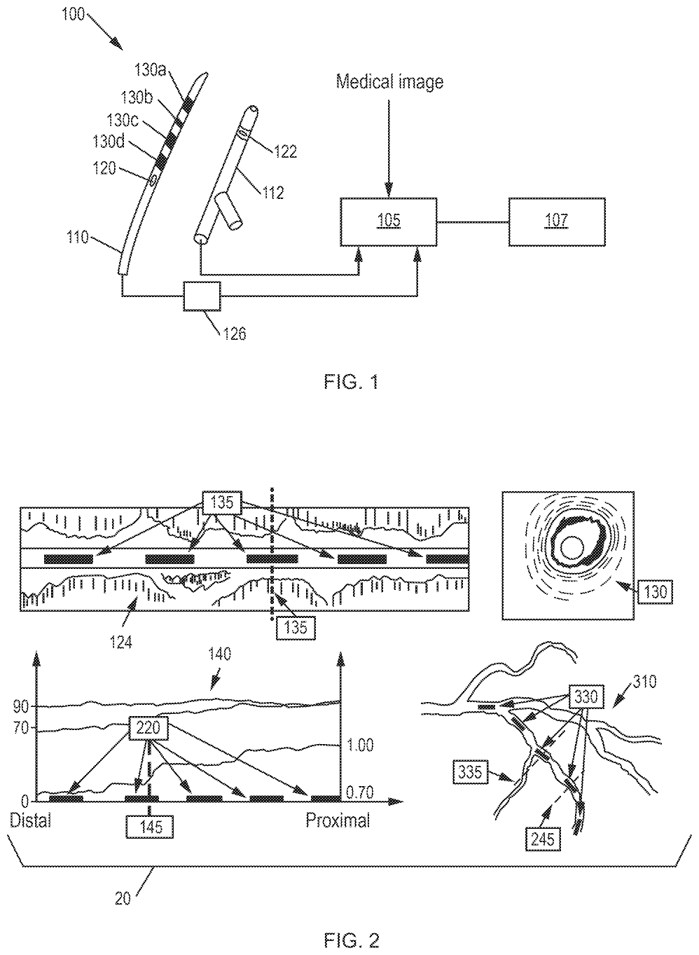

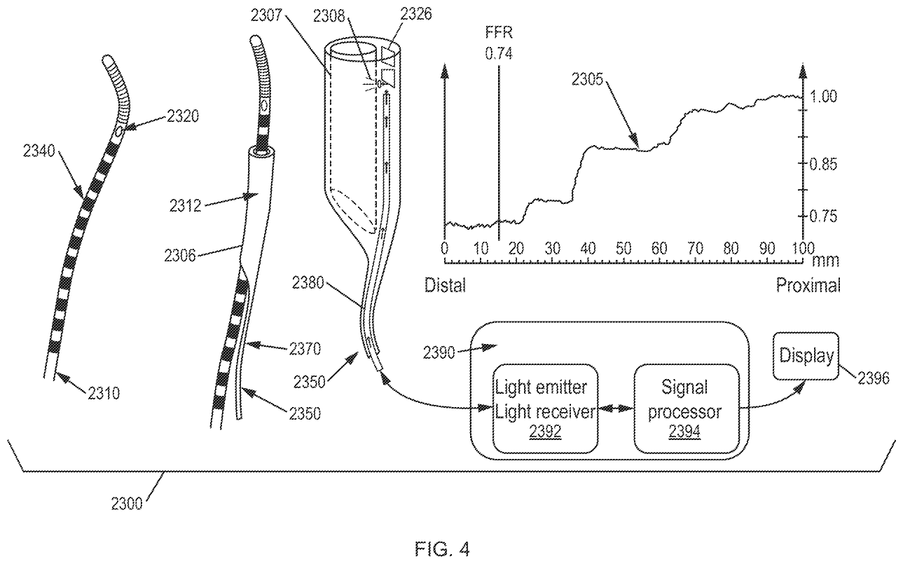

An example system for locating a medical device in a body lumen and providing for treatment delivery position detection is shown in FIG. 1. The system 100 includes a first flexible elongate instrument 110 and a second flexible elongate instrument 112 configured for parallel, relative movement with respect to the first flexible elongate instrument. The first flexible elongate instrument includes a plurality of imaging markers 130a-130d, which can be, for example, radiopaque imaging markers. A location information sensor 120 can be disposed at the first flexible elongate instrument 110. For example, a location information sensor 120 can be disposed on or in the first flexible elongate instrument at a distal portion of the instrument, as illustrated in FIG. 1. Alternatively, or in addition, a location information sensor 120 can be disposed in a separate component of the system, for example, in a microcatheter engaged with one of the first and second elongate instruments (see, e.g., FIG. 4). Alternatively, or in addition, a location information sensor 122 can be disposed at the second flexible elongate instrument. As illustrated, the location information sensor 122 of the second flexible elongate instrument is disposed at a distal portion of the instrument; however, it can alternatively be disposed at a proximal portion or in a microcatheter.

The first flexible elongate instrument 110 can be, for example, a guidewire, a wire including a diagnostic sensor (e.g., an FFR wire), and/or a wire including a therapeutic device (e.g. an atherectomy wire). The second elongate instrument 112 can be, for example, a catheter (e.g., an IVUS or OCT catheter, a balloon delivery catheter, a catheter of a biopsy device or aspiration device, an endoscopic catheter, etc.).

In embodiments, methods of locating a medical device in a body lumen and providing for treatment delivery position detection can include use of two or more types of second elongate instruments, for example, a diagnostic instrument and a treatment instrument.

The second elongate instrument 112 can be or can include a therapeutic delivery device. Therapeutic delivery devices can provide for delivery of a therapeutic device (e.g., a stent) or a therapy (e.g., ablation). Examples of therapeutic devices that can be used with the system include angioplasty devices, stents, embolization devices, atherectomy devices, ablation devices, drug-delivery devices, optical delivery devices, aspiration devices, and other devices capable of delivering a mechanical or physical intervention, a chemical intervention, and/or an energy-delivery intervention.

The system can further include a treatment activation sensor 126 disposed at or in operative arrangement with at least one of the first and second flexible elongate instruments 110, 112. The treatment activation sensor can be configured to detect an activation energy associated with delivery of a therapy or deployment of a therapeutic device by the second flexible elongate instrument (or a therapeutic delivery device thereof).

As used herein, an “activation energy” means energy applied in a treatment (e.g., illumination for light therapy) or energy applied for deployment of a treatment device (e.g., pressure to inflate a balloon for application of a stent). Examples of activation energy that can be applied in an endoluminal treatment procedure include pressure, ultrasound, infrared and near infrared light, and acoustic shockwaves.

As used herein, a “treatment activation sensor” means a sensor configured to detect a type of activation energy employed in the delivery of a treatment. Examples of treatment activation sensors include a pressure sensors, temperature sensors, optical sensors, electromagnetic sensors, mechanical sensors, thermal sensors, radiation sensors, chemical sensors, and biosensors.

The system further includes a processor 105 and a display 107. The processor 105 can receive at least one medical image that includes the first flexible elongate instrument 110 disposed in a body lumen. In addition, or alternatively, the medical image can be received by a separate system processor and independently displayed. The processor can be configured to establish a reference coordinate system based on the plurality of imaging markers 130a-d, which are visible in the medical image. The processor can receive therapeutic delivery information at a plurality of locations in the body lumen from a location information sensor 120, 122 and can correlate the therapeutic delivery information with an activation energy detected by the sensor 126 to determine a position of the delivered therapy within the body lumen based on the reference coordinate system. Optionally, the processor can receive diagnostic scan information at the plurality of locations of the body lumen from the first or second flexible elongate instrument. The processor can be further configured to correlate the diagnostic scan information with the imaging markers for the plurality of locations based on the reference coordinate system and location information as sensed by the location information sensor. The medical image can be, for example an X-ray image, such as an X-ray angiography image, such as one obtained during fluoroscopy.

As used herein, the term “medical image” is intended to include any image produced by a medical imaging system for the viewing of internal anatomy of a patient. Medical images can be obtained from, for example, magnetic resonance (MR) imaging, nuclear magnetic resonance (NMR) imaging, computed tomography (CT), X-ray, and positron emission tomography (PET), among other imaging modalities. A medical image can include one or more static images. For example, a medical image can be an ultrasound video.

As used herein, the term “X-ray image” is intended to include any image produced by X-rays being passed through a body, including, for example, an X-ray angiography image, an X-ray fluoroscopy image, and a computed tomography (CT) image. An “X-ray image” can include one or more static images. For example, an “X-ray image” can be an angiography video or fluoroscopy video comprising a plurality of images.

While the system 100 is generally described with regard to radiopaque markings and X-ray images, the system 100 can alternatively provide for use with other imaging modalities, including, for example, magnetic resonance (MR) imaging, nuclear magnetic resonance (NMR) imaging, and positron emission tomography (PET). For such modalities, the markings 130a-d can be modality-specific markers. For example, the markings 130a-d can comprise an MR-sensitive or NMR-sensitive (e.g., comprises atoms with a free nuclear spin), electromagnetic sensitive, electromechanical sensitive, optically sensitive, and/or mechanically sensitive material that is detectable or distinguishable in the image. Instead of an X-ray image, an MR, NMR, or PET image, among other modalities, can be obtained by the processor 105 for correlation with the diagnostic scan or therapeutic delivery information.

As used herein, the term “reference coordinate system” includes one-dimensional, two-dimensional, and three-dimensional spatial reference systems in which at least one location (typically an initial location) of the first flexible elongate instrument is registered with respect to the imaging markers, which are visible on a medical image, and upon which subsequent positions of the first or second flexible elongate instrument are determined. Examples of establishing 1D, 2D and 3D reference coordinate systems to provide for location determination during an endoluminal diagnostic scan or therapeutic intervention are further described in Pub. No. WO 2022/126101, the entire teachings of which are incorporated herein by reference. For example, establishing a 1D reference coordinate system can include registering an initial location of a flexible elongate instrument in the vessel with respect to the imaging markers. For a further example, establishing a 2D or 3D reference coordinate system can include generating a model of the imaging markers and, optionally, the vessel lumen, based on a representation of the imaging markers in one or more medical images (e.g., one or more X-ray angiogram images).

As used herein, “diagnostic scan information” includes any information obtained during a diagnostic scan, including, for example, information pertaining to a location of a diagnostic sensor, a reading by a diagnostic sensor, and an image obtained by a diagnostic device.

As used herein, “therapeutic delivery information” includes any information obtained during delivery of a therapeutic intervention, including, for example, information pertaining to a location of a therapeutic device, deployment of a therapeutic device, and delivery of a therapeutic treatment.

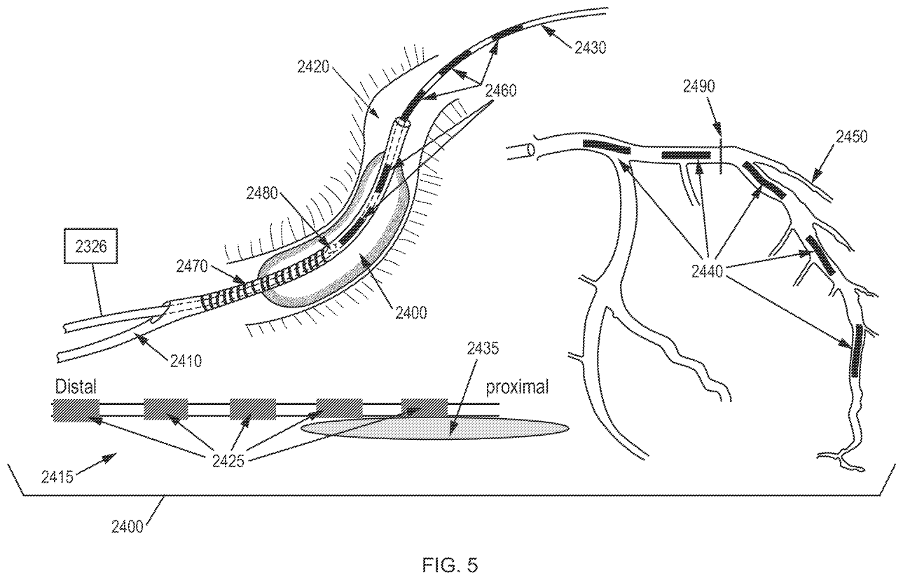

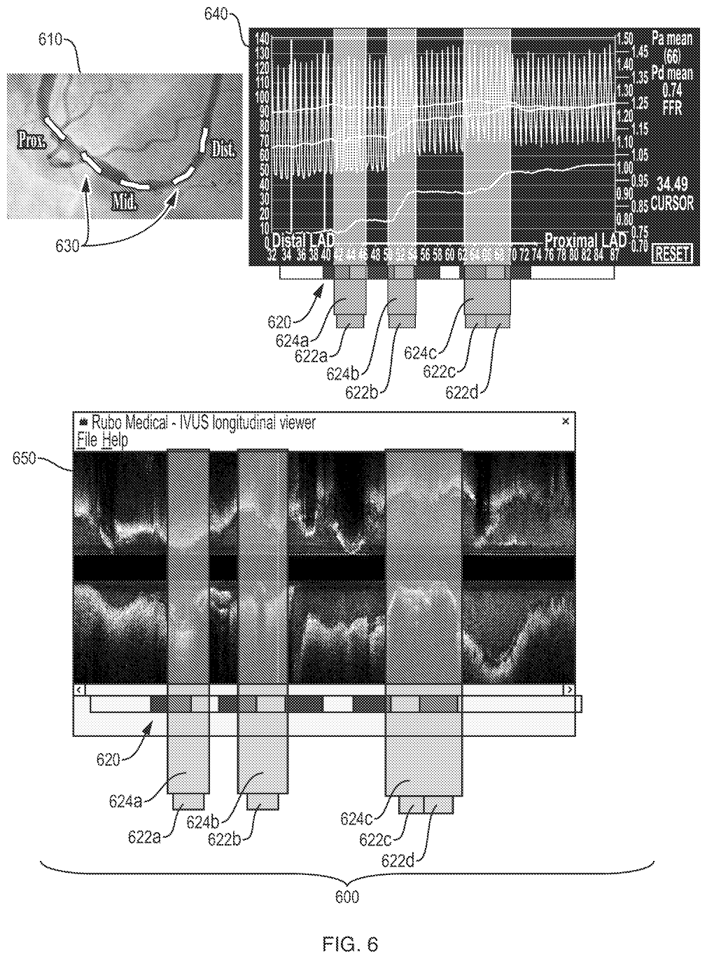

The display 107 is configured to display a composite image comprising the correlated diagnostic scan or therapeutic delivery information and the imaging markers. The composite image can be, for example, an image or graph obtained from the diagnostic scan, such an OCT image or an FFR graph, on which a representation of the imaging markers is superimposed (see, e.g., display 124, 140 of FIG. 2, display 2415 of FIG. 5, displays of FIG. 6). The composite image can be, in another example, the X-ray image on which a representation of a location of the diagnostic or therapeutic device is superimposed (see, e.g., display 310 of FIG. 2, display 2450 of FIG. 5, FIG. 6). The composite image can, in a further example, include an image in which information from multiple modalities or of multiple device positions are indicated (see, e.g., FIG. 2, FIG. 4, FIG, 5, FIG. 6). The composite image can include a representation of the body lumen in which the first and, optionally, second flexible elongate device is disposed and an indicator of a location of the device(s).

A flexible elongate instrument (e.g., flexible elongate instrument 110) having a plurality of radiopaque markers visible on an X-ray angiographic image of a vessel can advantageously provide for fixed points along the body lumen from which a location reference system (e.g., a linear location reference system, or a three-dimensional location reference system) can be defined. The location reference system can enable location correlation among the X-ray angiography image, diagnostic scan images or graphs, and/or therapeutic delivery devices. The flexible elongate instrument can remain at a same position in the body lumen such that subsequent positioning of additional flexible elongate instruments within the body can be correlated with or without real-time X-ray angiography. The methods and systems described herein can advantageously provide for significant reductions in X-ray exposure as compared with typical PCI procedures while enabling precise position detection of instruments within the body lumen and precise treatment delivery detection.

FIG. 2 illustrates an example display 20 of images obtained from an endoluminal procedure that includes IVUS and FFR scans, with the benefit of a co-location system. A flexible elongate instrument, such as the flexible elongate instrument 110 (FIG. 1) is disposed within a vessel, and an X-ray angiography image 310 is obtained that includes a visualization of the radiopaque markers 330 of the instrument. The locations of the markers in relationship to the vessel (as detected by the X-ray angiographic image) are projected onto the longitudinal IVUS pullback scan view 124 as markers 310 and onto the longitudinal FFR pullback scan view 140 as markers 220.

The IVUS vessel scan location indicated by the dashed line 135 can now be correlated to an X-ray angiographic vessel location indicated by the dashed line 335. It can be determined that the IVUS vessel cross sectional view 130 is located at the position of dashed line 335 in the X-ray angiographic view.

Similarly, the FFR pullback scan location indicated by dashed line 145 can be correlated to a location indicated by the dashed line 245 on the X-ray angiographic vessel image 310.

Establishing a location reference system and detecting a relative position of the first and second flexible elongate instruments can be performed as provided in International Pub. No. WO 2022/126101, the entire teachings of which are incorporated herein by reference. For example, a location information sensor disposed on the first flexible elongate instrument (e.g., sensor 120) can be a signal emitter or transducer of a modality that is detectable by a sensor located at the second flexible elongate instrument (e.g., sensor 122, which can be a diagnostic sensor). For example, if the second flexible elongate instrument is an IVUS catheter, the location information sensor of the first flexible elongate instrument can be an ultrasound transducer. The detection of a signal emitted from the IVUS device as it traverses the location information sensor can provide for the registration of co-location information such that a precise position of the IVUS device with respect to the first flexible instrument (and within the location reference system) can be determined. An intravascular scan display can then be generated such that, on pullback of the IVUS device, a precise position of the IVUS device with respect to the imaging markers and an angiography image can be determined and displayed. Alternatively, or in addition, at least one of the first and second flexible elongate instruments can include markers (e.g., “encoding markers”) detectable by the location information sensor, which can provide for more precise device tracking.

For example, a location information sensor (e.g., sensors 120, 122) can be disposed on one of two flexible elongate instruments and configured to detect encoding markers disposed on the other of the two flexible elongate instruments. The encoding markers can be disposed at and detected at a distal portion of the flexible elongate instruments to provide for accuracy at the location of interest within a vessel. One of the two flexible elongate instruments can further include imaging markers to provide for correlation to an X-ray image.

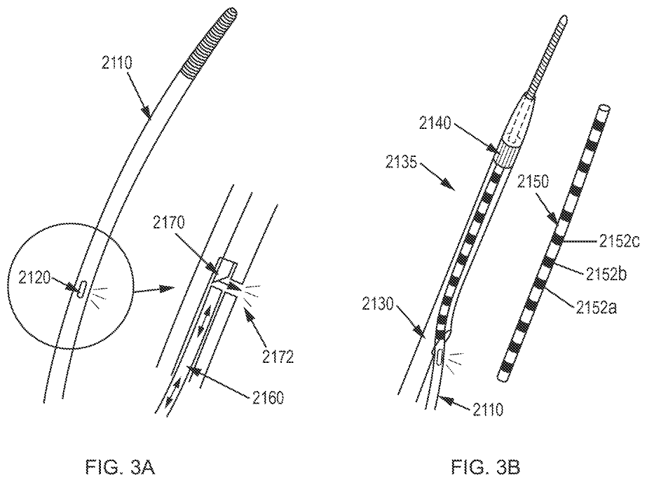

For example, as shown in FIG. 3A, a first flexible elongate instrument 2110 can be a guidewire (guidewire imaging markers not shown in FIGS. 3A and 3B for clarity) with an optical encoding sensor 2120 mounted at the distal portion of the flexible elongate instrument. The guidewire can be used in conjunction with a second flexible elongate instrument 2130 which, as illustrated in FIG. 3B, is a phased array IVUS catheter that can generate body lumen morphology information when inserted in a body lumen. However, any catheter can be configured to be used with such a guidewire such that a displacement of the catheter relative to the guidewire can be measured and output to a processor/computer. The displacement information can be correlated to diagnostic body lumen information obtained at each diagnostic point. The phased array IVUS catheter 2130 includes a phased array acoustic transducer 2140 near its distal tip. The catheter includes portions with one or a plurality of displacement encoding markers and portions without displacement encoding markers, which can optionally be configured to be in a periodic order. A monorail portion 2135 of the IVUS catheter includes a liner 2150 that is marked with optical linear encoding. The liner 2150 can be disposed within the monorail portion of the IVUS catheter, such that, as the catheter traverses over the guidewire 2110, the optical encodings are detected by the sensor 2120.

As further illustrated in FIG. 3A, a displacement signal can be transmitted through an optical fiber 2160. The guidewire 2110 can include, for example, a 45-degree polished fiber termination 2170 with a reflective coating configured to divert light from the fiber towards an aperture 2172 and to divert light reflected back to the aperture 2172 from the encoding markers, down the optical fiber to a light intensity meter. The encoding sensor 2120 detects an encoding signal from the inner diameter surface of the monorail liner and sends the signal to a processor for conversion to displacement information. In a simplified example implementation, when there is relative movement between the guidewire and the catheter, the optical encoding sensor can detect changes in reflected light intensity from encoding markings of different reflectance at specified intervals. A processor (e.g., processor 105) can be configured to count changes in signal intensity, from which a displacement between the IVUS catheter and the guidewire can be established.

Optionally, each of the displacement encoding markers 2152a-c can comprise a different color (e.g., red, green, and blue (RGB)) or a different greyscale intensity, with white light illumination from the optical transducer 2120, and an RGB-sensitive or greyscale-sensitive detector. Such an implementation has the advantage of providing different reflected signal time patterns, which can enable automatic direction detection. While the sensor 2120 is shown and described to be an optical sensor, other sensing modalities can instead be used (e.g., magnetic), as further described in Intl. Pub. No. WO 2022/126101. Encoding markings can be positioned on either an outer diameter surface or an inner diameter surface of a flexible elongate instrument. Optionally, displacement encoding markers on an outer diameter surface can comprise a first pigment of a selected reflectance, and encoding markers on an inner diameter surface can comprise a second pigment of a different selected reflectance. The different reflectance pigments can result in different reflectivity profiles. The displacement encoding markers can comprise a laser engraving such that micro-grooves of different depths are provided on the encoding surface. For example, a deeper groove can result in a decreased reflection intensity as compared with a shallower groove.

FIG. 4 depicts an example system 2300 that includes an encoding detector as a location information sensor. As illustrated, a first flexible elongate instrument 2310 is an FFR wire with a blood pressure sensor 2320 at the distal portion of the device and a section that is marked with optical encoding 2340. Radiopaque markings can also be included on the instrument 2310 (not shown in FIG. 4 for clarity). A second flexible elongate instrument 2350 includes an encoding reading catheter 2306, such as a micro-catheter, with an optical encoding sensor 2308 mounted at an inner surface of its guidewire lumen 2307 and facing the guidewire when the guidewire is inserted.

The reading catheter 2306 can be constructed with a short and low-profile, over-the-wire section 2312 to minimize interruption to blood flow, and a long shaft section 2370 that contains an optical fiber 2380, which is connected to a subsystem 2390 that includes a light emitter and light intensity meter 2392 and a signal processer 2394. The system 2300 can further include a display 2396 configured to display FFR ratio versus displacement distance, as shown in graph 2305. The subsystem 2390 can be included in a system (e.g., system 100) for co-location registration with, for example, an x-ray angiogram comprising imaging markers of the wire 2310.

The FFR wire 2310 can first be inserted into a coronary vessel and advanced to a location of interest. The reading micro-catheter 2306 can then be inserted over the FFR wire and follow the FFR wire until the encoding sensor 2308 reaches the region comprising encoding markers 2340 on the FFR wire near the location of interest. The micro-catheter can be held stationary relative to the vessel. During an FFR diagnostic vessel scan, the FFR wire can be pulled back in the coronary vessel while obtaining blood pressure readings, and the encoding sensor can provide an encoding signal to the signal processor, which translates the encoding signal to distance displacement. For example, the reading catheter can be held stationary at a coronary vessel location that is just proximal of the coronary ostium, which can provide for minimal disturbance to coronary blood flow.

While the system in FIG. 4 is illustrated with one of the flexible elongate instruments being an FFR wire, the instrument 2310 can instead be a therapeutic delivery device, such as an ablation device, an optical delivery device, aspiration device, etc. The reading catheter 2306 can include a sensor 2326 configured to detect activation of a treatment delivered by the device 2310. For example, the sensor 2326 can be a pressure sensor, temperature sensor, optical sensor, etc. as further described herein.

The reading catheter can be in operative arrangement with another device, such as a treatment delivery catheter (e.g., catheter 2410), such that advancement of the treatment delivery catheter to a treatment location can be detected and measured by the micro-catheter.

In the illustrated examples, the encoding sensor is disposed on or in a guidewire (as a first flexible elongate instrument) and the encoding markers are disposed on or in the catheter or in a guidewire lumen liner within a catheter (as a second flexible elongate instrument). However, positioning of the encoding sensor and encoding markers can vary. For example, because movement between the two flexible elongate instruments is relative, a measurement can be obtained if the guidewire is configured to provide the linear encoding and the catheter is configured to include a sensor with which an encoding signal can be detected.

FIG. 5 illustrates another example system 2400 providing for location determination of a therapeutic device. As illustrated, a first flexible elongate instrument 2430 is a guidewire having a plurality of radiopaque imaging markers 2460 positioned in a vessel lumen 2420 at a location of interest. A second flexible elongate instrument 2410 is catheter on which an angioplasty balloon 2400 is mounted.

A length of each of the radiopaque imaging markers 2460 and the distances between each of the imaging markers is known. A location of the angioplasty balloon 2400 can be measured relative to the vessel markings 2440 in a depiction of an angiographic X-ray image 2450 of the vessel lumen capturing the plurality of radiopaque markers 2460. The angiographic image 2450 need not be a real-time image, and the X-ray imager does not need to be on and emitting X-rays to determine a location of the balloon 2400 with respect to the image 2450. The angiographic image 2450 can be obtained with the guidewire 2430 inserted in the blood vessel 2420 such that both the plurality of imaging markers 2460 and the blood vessel 2420 can be identified in the image. Optionally, a plurality of X-ray angiographic body lumen images can be obtained from different angles, with the guidewire remaining at the same body lumen location, which can advantageously provide for 3D modelling of the vessel and instruments within the vessel.

The angioplasty balloon catheter 2410 includes encoding 2470 (e.g., optical markers) positioned proximal to the balloon at a selected distance. An encoding sensor 2480 is affixed to or included in the guidewire, which is at a selected distance from the plurality of imaging markers 2460. A relative position between the angioplasty balloon on the catheter and the plurality of markers on the guidewire can therefore be known when the encoding sensor 2480 first engages with the encoding 2470 on the angioplasty catheter. This position is referred to as the first engagement position, as shown in the figure. The short line 2490 appearing in the x-ray image 2450 depicts the location of the distal end of the balloon when the balloon catheter is at the engagement position with the guidewire. Once an angiographic image of the vessel is obtained with the positions of the plurality of imaging markers along the vessel identified in the image, a location of the angioplasty balloon in the vessel at the first engagement position can be measured. The vessel location of the angioplasty balloon can be continuously measurable thereafter, provided the encoding sensor remains within the encoded region of the angioplasty balloon catheter. The location of the angioplasty balloon in the vessel can be displayed in real-time in a linear fashion as shown by display 2415, for example, in which a simulated depiction of the vessel markings 2440 appear as markings 2425 and a simulated depiction of the balloon 2400 appears as balloon marking 2435. The representation of the balloon 2400 can be dimensionally scaled with respect to the vessel lumber to represent a true indication of its overall position.

If the encoding sensor 2480 moves out of range of the encoding 2470, a location of the balloon can be re-acquired when the encoding sensor re-engages with the encoded region. The balloon can stay within the length of the plurality of radiopaque imaging markers when the encoding sensor is within the length of the encoded region to maximize a range that the plurality of imaging markers can provide as an aid for vessel location correlation.

When tracking and displaying the angioplasty balloon location relative to the position of the plurality of imaging markings is performed in real-time or about real-time, the imaging markings can be used to correlate the balloon position in the vessel image in the angiography for its navigation rather than using real time X-ray imaging to reduce radiation exposure.

The system further includes a sensor 2426, such as a pressure sensor, which can be included in or otherwise associated with one of the flexible elongate instruments. Alternatively, the pressure sensor can be disposed at an inflator for the balloon 2400 and in operative arrangement with a processor (e.g., processor 105) of the system to provide a pressure reading. The sensor 2426 can detect a change in pressure or a pressure above a defined threshold that indicates inflation of the balloon 2400. On detection of inflation, a precise position of the deployment of the balloon can be determined and captured based on as associated timing of the pressure reading.

Several endoluminal procedures involve inflation of a balloon or other device for delivering a therapy. For example, inflators for balloons and catheters can be applied in cardiovascular procedures involving angioplasty, atherectomy, stent delivery, and lithotripsy. A treatment activation sensor can be included in a system to detect one or more pressures applied by an inflator or otherwise initiated by a device (e.g., shockwave devices) deploying a treatment.

The inclusion of treatment activation sensing (e.g., pressure readings) can provide for automatic registration of a treatment location. For example, a system can be configured to automatically record a treatment location (with respect to a reference coordinate system) upon detection of a pressure above a given threshold.

The inclusion of treatment deployment measurements (e.g., pressure) can also provide for a higher resolution location registration of the treatment with respect to other data acquired for a patient. For example, while a location of a therapeutic delivery device can be accurately recorded with high resolution using the systems and methods described in Intl. Pub. No. WO 2022/126101, the therapeutic delivery device may be permitted to move or caused to move to some extent near the intended treatment location prior to or during delivery of the treatment. As such, an exact location of the delivered therapy may be unknown or not precisely correlated to other patient data. The detection of pressure (or other energy providing for activation of a therapy) can advantageously provide timing information for correlation with the detected location information to produce a more accurate location measurement of the provided treatment. Such measurements can also enable improved correlation to other data associated with the patient, such as diagnostic data obtained prior to treatment or obtained in follow-up visits, and/or for subsequent procedure steps (e.g., delivery of a stent following angioplasty).

For example, as shown in the dataset 600 of FIG. 6, an X-ray angiography image 610 is obtained that includes a visualization of imaging markers 630 of a flexible elongate instrument (e.g., instrument 110) disposed within a vessel. Locations of the markers 610 in relationship to the vessel (as detected by the X-ray angiographic image) are correlated to and projected onto an FFR scan 640 and an IVUS scan 650 as markers 620. One or more treatment activation sensors can provide for correlation of an applied treatment to the patient data. For example, labels 624a, 624b, 624c can provide for an indication of stent locations, and labels 622a, 622b, 622c, and 622d can provide an indication of locations at which balloons were deployed within the vessel. Parameters associated with treatment can be included in the labeling and correlated with the patient data (e.g., a size of the stent, a size of the balloon, an applied pressure, etc.). For example, as shown with respect to the IVUS scan 650, it can be seen that label 624c is larger than labels 624a and 624b, where a larger stent was applied and where two balloon deployments were provided (as indicated by labels 622c, 622d).

The system can thus provide for auto-generation of datasets with precisely correlated diagnostic and treatment information. The information can include, for example, a type of device or tool used in the treatment, co-registered vessel location information, treatment parameters (e.g., pressure, duration, etc.), etc.

Higher resolution data resulting from the inclusion of pressure sensing and/or the sensed pressure measurement(s) itself (or other types of activation energy sensing) can be used to better inform subsequent treatment decisions. For example, with the addition of pressure-labeled data, artificial intelligence models can not only make use of higher fidelity data for the prediction of the success of a given treatment but can also provide predictions that can inform treatment parameters (e.g., a length of stent to be provided, an adequate pressure for application of the stent, etc.).

With such models and co-location systems, one or more pre-intervention imaging steps can be omitted for some patients. For example, a clinician can locate a treatment device for delivery of a therapy based on a prior-obtained diagnostic scan. Furthermore, therapeutic delivery steps can be optimized based on such models, with treatments accurately delivered to desired treatment locations. For example, a clinician can be provided with labeling resulting from a diagnostic scan and generated as a result of the model to indicate an extent of calcification and/or that calcification is such that a particular therapy is recommended for the location.

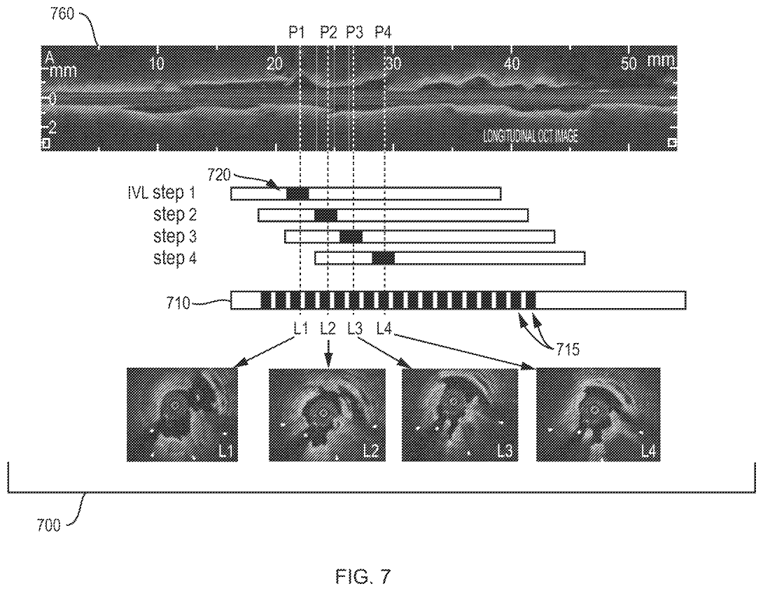

In another example, as shown in the dataset 700 of FIG. 7, an OCT imaging dataset 760 can be obtained with an encoded flexible elongate instrument 710 disposed within a vessel. The flexible elongate instrument 710 includes encoding markers 715 that can provide for precise correlation of the OCT data 760 to a location of a treatment delivery device. In the illustrated example, a second flexible elongate instrument (e.g., instrument 112) is an intravascular lithotripsy (IVL) device.

An IVL device is a lesion modification device that delivers energy to break superficial and deep calcium deposits at targeted locations or micro-regions of a vessel to facilitate adequate stent expansion during an interventional procedure, such as a percutaneous coronary intervention (PCI).

Typically, an IVL procedure is guided with angiogram and/or intravascular imaging, such as IVUS and OCT. Using OCT imaging as an example, as illustrated in FIG. 7, calcification areas and locations are identified in longitudinal and cross-sectional OCT images at positions P1, P2, P3 and P4. With position sensing of the first and second flexible elongate instruments provided by the described systems, an IVL device can be delivered to the intended treatment locations by aligning the device's emitter to those positions labeled as L1, L2, L3 and L4. The locations treated by the IVL device can be determined and correlated to the OCT data with use of a treatment activation sensor. For IVL, a device typically emits electric energy that then generates acoustic energy. In particular, a lithotripsy emitting produces electric sparks to create cavitation bubbles resulting in acoustic pressure waves to crack calcium inside the vessel. A treatment activation sensor for lithotripsy can be one that detects an electric discharge and/or pressure.

For example, starting with location (L1), an IVL balloon can be expanded under a desired pressure (Pa1). The IVL emitter can deliver energy to crack calcium in the vessel, which energy can be characterized by a pulse number (N1) and pulse time (t1). The IVL device can move to subsequent locations (L2, L3 and L4), at each of which the IVL emitter delivers energy controlled by pressure Pa, pulse number N, and time t for other lesions. A labeled dataset comprising pressure, pulse number, and time (e.g., (Pa1, N1, t1) (Pa2, N2, t2), (Pa3, N3, t3) and (Pa4, N4, t4)) with precise treatment correlation to other imaging datasets (e.g., angiogram and OCT, as illustrated) can be generated. Depending upon the nature of calcification and treatment objectives, multiple locations may be treated by IVL. Treatment parameters such as balloon inflation pressure, pulse cycle, pulse time, etc. can be included and co-registered with other imaging modalities. Labeled IVL treatment datasets can be automatically generated. For new cases, a predictive model for treatment optimization based on AI can be trained, at least in part, based on such diagnostic/treatment labeled datasets.

The systems described herein can provide for data acquisition, modeling, procedure guidance, precision lumen location correlation, and position information display with minimum radiation, as further described in Intl. Pub. No. WO 2022/126101, the entire teachings of which are incorporated herein by reference. The systems described herein improve upon those described in Intl. Pub. No. WO 2022/126101 with the inclusion of additional features providing for treatment sensing, treatment labeling with respect to co-registered imaging datasets, machine learning for treatment predictions and recommendations, and treatment guidance.

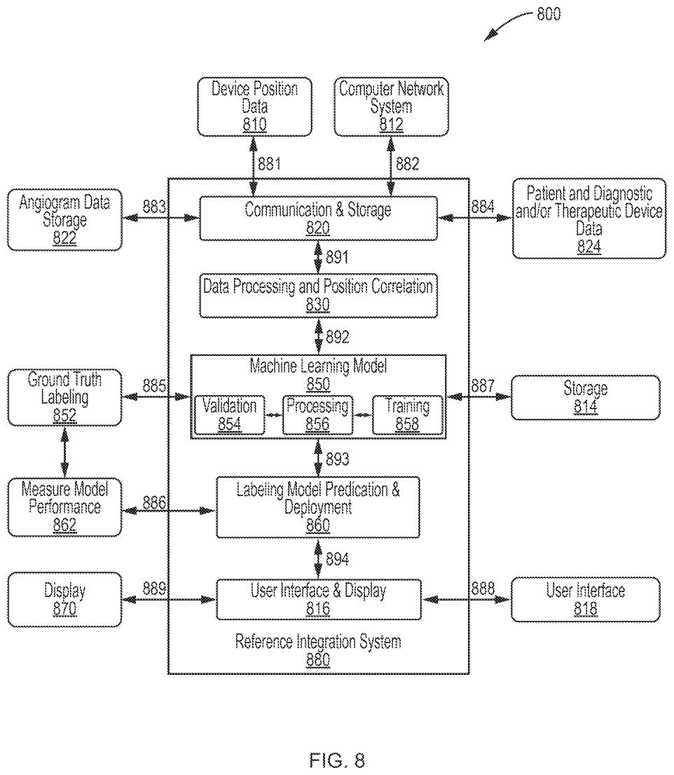

FIG. 8 is a block diagram illustrating an example system 800. A Reference Integration System 880 can include several subsystems: (1) Communication and Storage subsystem (820); (2) Data Processing and Position Correlation subsystem (830); (3) Machine Learning Model subsystem (850); (4) Labeling Model Prediction & Deployment subsystem (860); and (5) User Interface and Display (816).

The Communication and Storage subsystem (820) can interface with external data streams 881, 882, 883, and 884, store raw data on system memory banks, and provide an internal data stream 891, allowing the Data Processing and Position Correlation subsystem (830) to access different streams of data, save the processed data in the system memory bank, and interface with external storage as needed.

The Device Position Data interface (810) can interface with a guidewire, therapeutic device and/or diagnostic device to obtain position information and activation sensing input (881) and store corresponding data in the system memory bank for processing by a Data Processing and Position Correlation subsystem (830).

The Computer Network System interface (882) can transmit and/or receive data from local and/or external network storage systems containing information for signal processing in the subsystems. The data can be real-time or about real-time, and can be acquired from different procedures and/or steps such as but not limited to ECG (Electrocardiogram), Doppler, FFR (Fractional flow reserve), FFR-CT (Fractional flow reserve-computed tomography), IVUS (intravascular ultrasound), and OCT (Optical coherence tomography). The Computer Network System (882) can provide for saving raw data and final processed data from memory banks to local and/or external storage systems for further data processing from other therapeutic and/or diagnostic systems.

The Angiogram Data Storage interface (883) can interface with the Angiogram Data Storage (822) to obtain real time and/or about real time Angiogram data and store the data in the system memory banks processing by the Data Processing and Position Correlation subsystem (830).

The Diagnostic and/or Therapeutic System Data interface (884) can access diagnostic and therapeutic information before, during, and after procedures from a Diagnostic and/or Therapeutic system (824), such as but not limited to ECG (Electrocardiogram), Doppler, FFR (Fractional flow reserve), FFR-CT (Fractional flow reserve-computed tomography), IVUS (intravascular ultrasound), and OCT (Optical coherence tomography) systems. The Diagnostic and/or Therapeutic System Data interface (884) can also access therapeutic and/or diagnostic device Position Data or portion thereof, which can be unique to the configuration of the diagnostic and therapeutic systems and devices, such as but not limited to catheter pullback distance (i.e., as obtained at the proximal end of the catheter via an apparatus as part of the diagnostic/therapeutic system), and therapeutic delivery information, including activation sensing (e.g., pressure sensing).

The Data Processing/Position Correlation subsystem (830) can serve several functions. From the angiographic information input (883), including images of the radiopaque markers, the subsystem can establish 2D and/or 3D models of the flexible elongate instrument inside the lumen with dimension information and relative position to the lumen. The subsystem (830) can receive position and/or displacement information pertaining to the therapeutic and/or diagnostic device in real-time or about real-time from any of: Device Position Data (810), Diagnostic and/or Therapeutic System Data (824), and Communication & Storage (820) via the interface (891). The subsystem (830) can integrate the position data with 2D and/or 3D models of the flexible elongate instrument and generating real-time or about real-time device position illustrations, including superimposition of the illustration with the 2D and/or 3D models. The subsystem (830) can also generate position correlation display data via real-time or about real-time data integration among the 2D/3D model, simulated device illustration(s), diagnostic and therapeutic system data, and Angiogram data. The subsystem (830) can also provide for input and processing of operator/physician-selected viewing options, such as 2D/3D, a projection of interest, viewing angles with device signals at any location, and/or other execution requests via the User Interface and Display subsystem (816). The subsystem (830) can integrate therapeutic delivery data, including the detection of activation of a therapy delivered, with the established 2D and/or 3D model, including superimposition of therapy locations in an illustration based on the 2D and/or 3D models and labeling of patient datasets with therapeutic delivery information (e.g., timing, location, applied pressure or other activation energy, device size, etc.).

The internal data interfaces (891, 892) can serve as an interface between the Communication and Storage subsystem (820) and the Data Processing/Position Correlation subsystem (830) and the Machine Learning Model subsystem (850). Raw data, which can include data from the Device Position Data interface (881), Computer Network System interface (882), Angiogram Data Storage interface (883), and Diagnostic and/or Therapeutic System Data interface (884), can reside in local memory bank within the subsystems.

The Machine Learning Model subsystem (850) can receive processed, location-correlated diagnostic and/or therapeutic data via an internal data interface (892) from the Data Processing and Position Correlation subsystem (830). The Machine Learning Model subsystem can include modules providing for the training of a machine learning model to associate delivered therapies and, optionally, patient outcomes, therapeutic device(s) used (such as different types of drug eluting stents), procedural specifics (such as a type of guidewire, technique(s) for crossing lesions), preprocedural imaging (such as cardiac CT or CT angiography), with diagnostic patient data (such as family history and genetics) and other therapeutic patient data. The modules can include a Validation module (854), a Processing module (856), and a Training module (858). The Machine Learning Model (850) can communicate via an interface (885) to obtain Ground Truth Labeling (852) input and can communicate via interface (887) with a Storage subsystem (814), which can provide for storage of labeled datasets and other associated data.

An internal data interface (893) can serve as an interface between The Machine Learning Model subsystem (850) and the Labeling Model Prediction and Deployment subsystem (860). The Labeling Model Prediction and Deployment subsystem (860) can provide several functions, including providing for data communication via interface (886) to present and obtain Model Performance Measurements (862), which can provide for and/or be combined with Ground Truth Labeling (852) for the training of the machine learning model. The Labeling Model Prediction and Deployment subsystem (860) can provide for treatment recommendations and/or predictive outcomes based on a given patient's diagnostic data and processing (e.g., by Processing module (856)) with respect to the Machine Learning Model (850).

To provide for training of the machine leaning model, the Training module (858) can associate Ground Truth Labeling data (852) with patient datasets comprising diagnostic and therapeutic delivery data, and the Validation module (854) can provide for validation of treatment recommendations and predictions. Data communication from the Labeling Model Prediction and Deployment subsystem (860) to and from the User Interface & Display subsystem (816) can occur via an internal data interface (894).

The User Interface and Display subsystem (816) can place the processed data (892, 893, 894) from the Data Processing/Position Correlation subsystem (830), the Machine Learning Model subsystem (850) and/or the Labeling Model Prediction and Deployment subsystem (860) in a proper format (889) for display by the Display system (870), such as in a graphical representation based upon User Interface data inputs (888) from a User Interface device (818). Operator/physician interface inputs can be embedded into the display data (889) to display system (870) or can be embedded into the operator/physician interface data (888) to the User Interface (818) as a separated display and control.

The provided systems and methods can be applied to any interventional procedure for a body lumen. Optionally, a GUI can be rendered on the Reference Integration System 880 with components or controls to allow an operator to interact with the Reference Integration System 880 via command control for execution, including providing for interfacing a lumen position correlation display with third-party diagnostic and therapeutic systems. A form of the visualization display system (e.g., display 870) can vary and can be or include, for example, a monitor, mobile device, wearable device, and AR/VR head mounted device. The inputs from an operator/physician at an operator/physician interface 818 can be executed via an electronic device, such as a computer, a server with a monitor, a host workstation, a controller with a monitor, and a third-party system operator/physician interface. An I/O can include a keyboard, joystick, mouse, touch display, project device, microphone, any consumer and/or wearable electronics, such as mobile phone, AR headwear, pointing device, and audio feedback, for communicating with the Reference Integration System 880 for procedure control, data rendering and visual display, data storage, and basic data process functions. Such a connection mechanism can provide ease of use workflow with adequate customization flexibility on real-time or about real-time lumen position correlation and associated data processing steps for users throughout a guided procedure. The interface connections 881, 882, 883, 884, 885, 886, 887, 888, and 889 with the Reference Integration System 880 as shown in FIG. 8 can be established via various connection mechanisms such as cables, cell networks (4G, 5G), local and or wide area network (LAN and WAN), Bluetooth network or wireless.

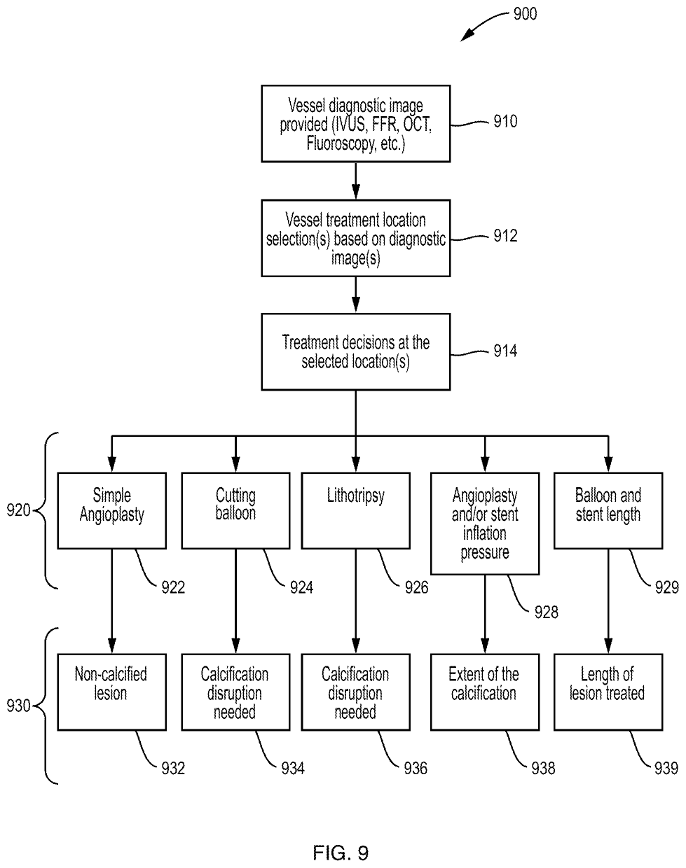

A flowchart illustrating an example workflow and example labeling for a machine learning workflow is shown in FIG. 9. The method 900 includes providing diagnostic image(s) of a vessel (910) and selecting at least one treatment location from the diagnostic image(s) (912). The selection of a treatment location can be performed by a physician or operator. Upon training of a machine learning subsystem, predicted/recommended vessel treatment locations can be identified by the system and confirmed or validated by a physician or operator.

The method further includes providing a treatment decision for each selected location (914). The treatment decision can be determined by a physician or operator. Upon training of a machine learning subsystem, predicted/recommended treatments can be identified by the machine learning subsystem and confirmed or validated by a physician or operator.

Example options for treatment decisions 920 are shown, including a simple angioplasty (922), deployment of a cutting balloon (924), lithotripsy (926), angioplasty and/or stent inflation pressure (928), and balloon and stent parameters (929), such as length.

With correlation to the vessel treatment location (as identified in item 912 and with features obtained from the diagnostic data/images), the determined treatment decisions 920 can provide for identification of lesion types and can be used in the predictive model to inform future treatment recommendations. Examples of labels 930 are shown. For example, the application of a simple angioplasty (922) to a particular vessel treatment location can indicate a non-calcified lesion (932) in that location. In further examples, application of a cutting balloon (924) and/or lithotripsy (926) can indicate a need for calcification disruption (934, 936); an angioplasty and/or stent inflation pressure can be associated with an extent of calcification (938); and balloon and/or stent lengths can be associated with lesion treatment lengths (939).

Such associations can be particularly helpful with respect to IVUS and OCT images, which can be difficult to interpret. With a database comprising such label data sets, artificial intelligence can be of a significant value in assisting a physician in interpreting IVUS and/or OCT images, as well as other imaging modalities, and derive a treatment decision.

In some embodiments, therapeutic delivery devices can move along an encoded guidewire positioned inside the vessel. The guidewire does not need to move while other catheters move along it. After a diagnostic procedure, the guidewire can be left in place inside the vessel to be used by other devices, including therapeutic delivery devices. When other devices are inserted and advanced along the guidewire, markers disposed on the guidewire can help clinicians guide the other devices to vessel features observed from intravascular scan. This is particularly useful because the markers are not only able to help guide another device to the vessel location on live X-ray (if used), but also can help guide another device to locations displayed on a longitudinal vessel scan image by correlating its position from, for example, a live X-ray back to the intravascular scan image.

A flexible elongate instrument can comprise, at least in part, one or more rigid portions or components. For example, a flexible elongate instrument can include or provide for the travel of a biopsy device or an aspiration device, which can include a rigid needle or other rigid structure(s) to effect obtaining a diagnostic sample or providing for delivery of a treatment.

As used herein, the term, “therapeutic and/or diagnostic device” refers to a region of a flexible elongate endoluminal instrument that is adapted to perform a function when inside of a body lumen. Examples of therapeutic and/or diagnostic devices on a flexible elongate endoluminal instrument include a stent, balloon, ablation tips, electrodes, ultrasound imaging transducer, pressure sensor, and optical coherent tomography light emitting tip.

As used herein, the terms “diagnostic device” or “diagnostic system” refers to medical equipment, medical systems, an instrument, or a component thereof, an apparatus or substance, either active or passive, that is used during medical procedures, including interventional procedures both inside and/or outside of the body, for the detection, analysis, and/or measuring of a disease or medical condition of a patient. A diagnostic device can, for example, measure a temperature, pressure, conductivity, density, blood flow rate, oxygen level, or tissue morphology of the lumen. Examples of diagnostic devices that can be used with the provided methods and systems include intravascular ultrasound (IVUS) devices, optical coherence tomography (OCT) devices, photoacoustic sensing devices, fractional flow reserve (FFR) devices, endoscopic devices, arthroscopic devices, biopsy devices, and other devices which include a sensor configured to measure a tissue composition, a physical property, a physiological property, and/or a molecular property of anatomy.

As used herein, the terms “therapeutic device” or “therapeutic system” refers to medical equipment, medical systems, an instrument or a component thereof, an apparatus or substance, either active or passive, that is used during medical procedures, including interventional procedures for the treatment of a disease or medical condition of a patient, and in the prevention of disease or condition, amelioration from a disease or condition, or maintenance or restoration of health. Examples of therapeutic devices that can be used with the provided methods and systems include angioplasty devices, stents, embolization devices, atherectomy devices, ablation devices, drug-delivery devices, optical delivery devices, aspiration devices, and other devices capable of delivering a mechanical or physical intervention, a chemical intervention, or an energy-delivery intervention.

A diagnostic and/or therapeutic device can be a guidewire, a microcatheter, a thrombectomy catheter, a steerable catheter, a balloon catheter, a device delivery catheter, a cardiac catheter, a renal catheter, a urinary catheter, an oncology catheter, a robotic catheter/guidewire, a biopsy device, an atherectomy device (which can include or exclude an aperipheral arterial disease catheter), a lithotripsy device, or a neuromodulation device. A cardiac catheter can include or exclude a radiofrequency ablation catheter, a mapping catheter, a percutaneous transluminal angioplasty (PTA) catheter, an embolic protection device, a chronic total occlusion device, an infusion catheter, a snare, a support catheter, a thermodilution catheter, and a valvulotome. A diagnostic and/or therapeutic device can be configured to be used in a body lumen which does or does not have blood flow.

As used herein, the terms “diagnostic scan” or “body lumen information scan” or “vessel displacement scan” refer to imaging or assessing part or all of a body lumen using a diagnostic device. A diagnostic scan can measure any of a pressure, temperature, density, conductivity, inductance, tissue morphology, etc. at selected locations across the body lumen.

As used herein, the term “radiopaque” refers to refers to opacity from the radio wave to X-ray portion of the electromagnetic spectrum. Radiopaque components serve as a contrast when viewed with X-rays. Radiopaque materials can be made from, for example, titanium, platinum, gold, palladium, tungsten, barium, zirconium oxide, or any material identified by ASTM F640 Standard Test Methods for Measuring Radiopacity for Medical Use, as of Oct. 1, 2020.

As used herein, the term “stent” refers to a tubing placed into a body lumen to keep a passageway open. Stents can be placed into, for example, a coronary lumen to treat a coronary disease, a cerebrovasculature lumen to treat a cerebrovascular disease, a peripheral lumen to treat a peripheral disease, a ureteral lumen to treat an ureteral disease, and a gastrointestinal lumen to treat a gastrointestinal tract disease.

As used herein, the term, “linear position” refers to a distance between two objects or two identified regions in a body lumen, as measured following the path of the body lumen. The shape of the line can thus be straight or curvilinear. A curvilinear line can comprise multiple curves. The term “linear position” is used to distinguish from the term “linear distance” which refers to a distance between the two selected objects or two identified regions.

Flexible elongate instruments can generally comprise a proximal end, a distal end, and at least one of a sensor and a plurality of elements circumferentially or partially circumferential positioned around the flexible elongate instrument. A sensor disposed on or in a flexible elongate instrument can be shaped and adapted for insertion into a body lumen. The elements can be imaging markers, displacement encoding markers, or both. The plurality of elements can be independently of a selected distance from each other, of a selected dimension (e.g., width), and/or of a selected shape. The width of the elements can range from 0.01 mm to 3 cm. The number of elements can range from 2 to 500. The number of elements can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. Optionally, the elements can provide for a checksum—for example, a width of three successive elements can equal a width of a fourth successive element.

As used herein, the term, “displacement encoding” refers to a region on a flexible elongated instrument that comprises a plurality of encoding elements (also referred to as “encoding markers”) positioned at selected distance intervals on the flexible elongate instrument. The encoding elements are detectable by an encoding sensor.

As used herein, the term “encoding sensor” refers to a device that can detect or measure the displacement encoding. The displacement encoding can be positioned to be located on a first flexible elongate instrument and the encoding sensor can be positioned to be located on a second flexible elongate instrument. When the encoding sensor is in proximity to the displacement encoding, the encoding sensor can detect one or a plurality of the encoding elements. The encoding sensor can, for example, comprise a transducer that can transmit and/or receive a physical signal. The physical signal can be optical, electrical, magnetic, inductive, or capacitive. Variations in the signal generated by the encoding sensor on a second flexible elongate instrument when the encoding sensor is in proximity to, and moving in a direction parallel to, the first flexible elongate instrument, can be used to measure the relative displacement of the encoding sensor on the second flexible elongate instrument relative to the encoded section on a first flexible elongate instrument.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A system for locating a treatment provided in an endoluminal procedure, comprising:
a first flexible elongate instrument comprising a plurality of imaging markers;
a second flexible elongate instrument configured for relative movement with respect to the first flexible elongate instrument and comprising a therapeutic delivery device;
a location information sensor disposed at the first flexible elongate instrument or at the second flexible elongate instrument;
an treatment activation sensor configured to detect an activation energy associated with delivery of a therapy by the therapeutic delivery device; and
a processor configured to:
establish a reference coordinate system based on the plurality of imaging markers, the plurality of imaging markers being visible in a medical image comprising the first flexible elongate instrument disposed in a body lumen,
receive therapeutic delivery information at a plurality of locations in the body lumen from the location information sensor, and
correlate the therapeutic delivery information with the detected activation energy to determine a position of the delivered therapy within the body lumen based on the reference coordinate system.

2. The system of claim 1, wherein the activation sensor is a pressure sensor.

3. The system of claim 2, wherein the therapeutic delivery device is an inflation device.

4. The system of claim 1, wherein the processor is further configured to:
receive diagnostic scan information at the plurality of locations in the body lumen from a diagnostic device; and
correlate the diagnostic scan information with the therapeutic delivery information and the detected activation energy to determine a position of the delivered therapy relative to the diagnostic scan information.

5. The system of claim 4, wherein receipt of the diagnostic scan information occurs prior to delivery of the therapy.

6. The system of claim 4, wherein the first flexible elongate instrument comprises the diagnostic device.

7. The system of claim 4, wherein the diagnostic device is configured for relative movement with respect to the first flexible elongate instrument.

8. The system of claim 1, wherein the therapy comprises an angioplasty, atherectomy, stent delivery, lithotripsy, or a combination thereof.

9. The system of claim 1, further comprising a display configured to display a composite image comprising a representation of the position of the delivered therapy for use with subsequent procedures.

10. A method for locating a treatment provided in an endoluminal procedure, comprising:
establishing a reference coordinate system based on a plurality of imaging markers of a first flexible elongate instrument, the plurality of imaging markers being visible in a medical image comprising the first flexible elongate instrument disposed in a body lumen;
determining therapeutic delivery information at a plurality of locations in the body lumen from a location information sensor disposed at the first flexible elongate instrument or at a second flexible elongate instrument, the second flexible elongate instrument configured for relative movement with respect to the first flexible elongate instrument and comprising a therapeutic delivery device;
detecting an activation energy associated with associated with delivery of a therapy by the therapeutic delivery device; and
correlating the therapeutic delivery information with the detected activation energy to determine a position of the delivered therapy within the body lumen based on the reference coordinate system.

11. The method of claim 10, wherein detecting the activation energy comprises detecting a pressure.

12. The method of claim 11, wherein the detected pressure indicates inflation of the therapeutic delivery device.

13. The method of claim 10, further comprising:
receiving diagnostic scan information at the plurality of locations in the body lumen from a diagnostic device; and
correlating the diagnostic scan information with the therapeutic delivery information and the detected activation energy to determine a position of the delivered therapy relative to the diagnostic scan information.

14. The method of claim 13, wherein receiving the diagnostic scan information occurs prior to delivery of the therapy.

15. The method of claim 13, wherein the first flexible elongate instrument comprises the diagnostic device.

16. The method of claim 13, wherein the diagnostic device is configured for relative movement with respect to the first flexible elongate instrument.

17. The method of claim 10, wherein the therapy comprises an angioplasty, atherectomy, stent delivery, lithotripsy, or a combination thereof.

18. The method of claim 10, further comprising displaying a composite image comprising a representation of the position of the delivered therapy for use with subsequent procedures.

19. A method for generating a treatment recommendation for an endoluminal procedure for a patient, comprising:
receiving diagnostic scan information at a plurality of locations in a body lumen of the patient, the diagnostic scan information correlated to a reference coordinate system established based on imaging markers of a flexible elongate instrument disposed in the body lumen and visible in a medical image of the body lumen;
applying a machine learning model to the diagnostic scan information to identify a prospective treatment location, the machine learning model calibrated using training data comprising diagnostic scan information and therapeutic delivery information, including detected activation information for the therapeutic delivery, for a set of patients; and
outputting a treatment recommendation for the patient based on the applied machine learning model, the treatment recommendation including a location for treatment identified with respect to the reference coordinate system.

20. A method for generating a treatment recommendation for an endoluminal procedure for a patient, comprising:
receiving diagnostic scan information at a plurality of locations in a body lumen of the patient, the diagnostic scan information correlated to a reference coordinate system established based on imaging markers of a flexible elongate instrument disposed in the body lumen and visible in a medical image of the body lumen;
applying a machine learning model to the diagnostic scan information to obtain a predictive label associated with one or more treatment parameters, the machine learning model calibrated using training data comprising diagnostic scan information and therapeutic delivery information, including detected activation information for the therapeutic delivery, for a set of patients; and
outputting a treatment recommendation for the patient based on the obtained predictive label.
